(19) United States
(12) Patent Application Publication
(10) Pub. No.: US 2023/0266318 A1
(43) Pub. Date: Aug. 24, 2023

(54) MICROSCOPIC DETECTION OF BLAST-TRANSFORMED MONONUCLEAR CELLS

(71) Applicant: IMMUNOGENETICS.COM, INC., Dublin, CA (US)

(72) Inventor: Robert Michael Williams, Dublin, CA (US)

(73) Assignee: IMMUNOGENETICS.COM, INC., Dublin, CA (US)

(21) Appl. No.: 18/112,142

(22) Filed: Feb. 21, 2023

Related U.S. Application Data
(60) Provisional application No. 63/313,047, filed on Feb. 23, 2022.

(51) Int. Cl. G01N 33/569; G01N 33/574; G01N 21/64

(52) U.S. Cl. CPC G01N 33/56972; G01N 33/57484; G01N 21/6428; G01N 2800/26; G01N 2800/52; G01N 2021/6441

(57) ABSTRACT

The present disclosure provides methods for determining prognosis of cancer and/or infection in a subject based on the number of viable blast-transformed mononuclear cells and/or the number of viable large granular lymphocytes in a blood sample obtained from the subject.

### CROSS-REFERENCE TO RELATED APPLICATIONS

This application claims priority to U.S. Provisional Patent Application Ser. No. 63/313,047, filed Feb. 23, 2022; the entire contents of which are herein incorporated by reference.

### TECHNICAL FIELD

The present disclosure relates to methods for assessing prognosis of cancer and/or infection in a subject.

### BACKGROUND

Determining the prognosis of cancer and infection in an individual may be crucial for developing successful treatment and/or screening plans for the individual. Thus, there is need in the art to develop prognostic assays for management, treatment, and screening of cancer and infection in subjects.

### SUMMARY

Provided herein are methods of determining prognosis of cancer or infection in a subject that include: (a) contacting a blood sample obtained from the subject with a cell viability dye; (b) after step (a), generating a microscopic slide using the blood sample; (c) identifying a number of viable blast-transformed mononuclear cells and/or a number of viable large granular lymphocytes in the microscopic slide using microscopic analysis; and (d) identifying a subject having an increased number of viable blast-transformed mononuclear cells and/or an increased number of viable large granular lymphocytes, as compared to a reference level, as having an improved prognosis of cancer or infection; or identifying a subject having a decreased number of viable blast-transformed mononuclear cells and/or a decreased number of viable large granular lymphocytes, as compared to a reference level, as having a poor prognosis of cancer or infection.

In some embodiments of any of the methods described herein, step (b) comprises one or more centrifugation steps. In some embodiments of any of the methods described herein, one of the centrifugation steps comprises the use of a Cytospin instrument. In some embodiments of any of the methods described herein, the microscopic slide is a permanent microscopic slide comprising a cover slip.

In some embodiments of any of the methods described herein, step (c) comprises identifying the number of viable blast-transformed mononuclear cells in the microscopic slide. In some embodiments of any of the methods described herein, step (c) comprises identifying the number of viable large granular lymphocytes in the microscopic slide. In some embodiments of any of the methods described herein, step (c) comprises identifying the number of viable blast-transformed mononuclear cells and the number of viable large granular lymphocytes in the microscopic slide.

In some embodiments of any of the methods described herein, the reference level is a 75^{th} percentile of the median level of the number viable blast-transformed mononuclear cells or the 75^{th} percentile of the median level of the number of viable large granular lymphocytes in a healthy patient population. In some embodiments or any of the methods described herein, the reference level is a 80^{th} percentile of the median level of the number viable blast-transformed mononuclear cells or the 80^{th} percentile of the median level of the number of viable large granular lymphocytes in a healthy patient population. In some embodiments of any of the methods described herein, the reference level is a 90^{th} percentile of the median level of the number viable blast-transformed mononuclear cells or the 90^{th} percentile of the median level of the number of viable large granular lymphocytes in a healthy patient population.

In some embodiments of any of the methods described herein, step (b) comprises the use of one or more fluorophore-labelled antibodies specific for one or more cell surface proteins. In some embodiments of any of the methods described herein, the one or more labelled antibodies bind specifically to an antigen selected from the group consisting of: CD3, CD4, CD5, CD8, CD11 c, CD14, CD16, CD19, CD20, CD25, CD27, CD28, CD38, CD39, CD45, CD45RA, CD45RO, CD56, CD57, CD62L, CD66b, CD94, 30 CD103, CD122, CD123, CD127, CD161, CD223, CD294, CCR4, CCR6, CCR7, CXCR3, CXCR5, IgA, IgD, IgE, IgG, IgM, TCRγδ, KIR, NKG2A, NKGD, NKp30, NKp44, NKp46, NKp80, CTLA-4, and GITR. In some embodiments of any of the methods described herein, the one or more labelled antibodies are one or more fluorophore-labelled antibodies.

In some embodiments of any of the methods described herein, before step (b), the method further comprises incubating the blood sample with an antigen specific for an infection for 6 hours to 3 days. In some embodiments of any of the methods described herein, the blood sample is incubated with the antigen for about 36 hours to about 60 hours.

In some embodiments of any of the methods described herein, the viable blast-transformed mononuclear cells are viable blast transformed T cells and/or viable blast transformed B cells. In some embodiments of any of the methods described herein, the viable large granular lymphocytes are NK cells.

In some embodiments of any of the methods described herein, the blood sample is a venous blood sample. In some embodiments of any of the methods described herein, the blood sample is a capillary blood sample. In some embodiments of any of the methods described herein, the capillary blood sample is a finger stick blood sample.

Some embodiments of any of the methods described herein further include generating a frozen blood sample from a portion of the blood sample. In some embodiments of any of the methods described herein, the blood sample is frozen in a solution comprising DMSO. In some embodiments of any of the methods described herein, the method comprises storing the frozen blood sample at a temperature of less than 0° C. for at least 1 day. In some embodiments of any of the methods described herein, the frozen blood sample is stored at a temperature of less than −80° C. for at least 1 day. In some embodiments of any of the methods described herein, the frozen blood sample is stored at a temperature of less than −190° C. for at least 1 day.

Some embodiments of any of the methods described herein further include thawing the frozen blood sample and performing additional immunological testing/analysis on the thawed blood sample.

In some embodiments of any of the methods described herein, the subject has not been identified or diagnosed as having a cancer or infection. In some embodiments of any of the methods described herein, the subject has previously been identified or diagnosed as having a cancer or infection.

In some embodiments of any of the methods described herein, the method comprises selecting for a subject identified as having an improved prognosis of cancer or infection a low frequency of cancer or infection screening for the subject.

In some embodiments of any of the methods described herein, the method comprises selecting for a subject identified as having a poor prognosis of cancer or infection a high frequency of cancer or infection screening for the subject.

Also provided herein are methods of selecting a treatment for a subject not identified or diagnosed as having cancer or infection that include: (a) contacting a blood sample obtained from the subject with a cell viability dye; (b) after step (a), generating a microscopic slide using the blood sample; (c) identifying a number of viable blast-transformed mononuclear cells and/or a number of viable large granular lymphocytes in the microscopic slide using microscopic analysis; (d) identifying a subject having an increased number of viable blast-transformed mononuclear cells and/or an increased number of viable large granular lymphocytes, as compared to a reference level, as having an improved prognosis of cancer or infection; or identifying a subject having a decreased number of viable blast-transformed mononuclear cells and/or a decreased number of viable large granular lymphocytes, as compared to a reference level, as having a poor prognosis of cancer or infection; and (e) selecting for a subject identified as having an improved prognosis of cancer or infection a low frequency of cancer or infection screening for the subject, or selecting for a subject identified as having a poor prognosis of cancer or infection a high frequency of cancer or infection screening for the subject.

In some embodiments of any of the methods described herein, step (b) comprises one or more centrifugation steps. In some embodiments of any of the methods described herein, one of the centrifugation steps comprises the use of a Cytospin instrument. In some embodiments of any of the methods described herein, the microscopic slide is a permanent microscopic slide with a cover slip.

In some embodiments of any of the methods described herein, step (c) comprises identifying the number of viable blast-transformed mononuclear cells in the microscopic slide. In some embodiments of any of the methods described herein, step (c) comprises identifying the number of viable large granular lymphocytes in the microscopic slide. In some embodiments of any of the methods described herein, step (c) comprises identifying the number of viable blast-transformed mononuclear cell and the number of viable large granular lymphocytes in the microscopic slide.

In some embodiments of any of the methods described herein, the reference level is a 75th percentile of the median level of the number viable blast-transformed mononuclear cells or the 75th percentile of the median level of the number of viable large granular lymphocytes in a healthy patient population. In some embodiments of any of the methods described herein, the reference level is a 80th percentile of the median level of the number viable blast-transformed mononuclear cells or the 80th percentile of the median level of the number of viable large granular lymphocytes in a healthy patient population. In some embodiments of any of the methods described herein, the reference level is a 90th percentile of the median level of the number viable blast-transformed mononuclear cells or the 90th percentile of the median level of the number of viable large granular lymphocytes in a healthy patient population.

In some embodiments of any of the methods described herein, step (b) comprises the use of one or more fluorophore-labelled antibodies specific for one or more cell surface proteins. In some embodiments of any of the methods described herein, the one or more labelled antibodies bind specifically to an antigen selected from the group consisting of: CD3, CD4, CD5, CD8, CD11c, CD14, CD16, CD19, CD20, CD25, CD27, CD28, CD38, CD39, CD45, CD45RA, CD45RO, CD56, CD57, CD62L, CD66b, CD94, CD103, CD122, CD123, CD127, CD161, CD223, CD294, CCR4, CCR6, CCR7, CXCR3, CXCRS, HLA-DR, IgA, IgD, IgE, IgG, IgM, TCRγδ, KIR, NKG2A, NKGD, NKp30, NKp44, NKp46, NKp80, CTLA-4, and GITR. In some embodiments of any of the methods described herein, the one or more labelled antibodies are one or more fluorophore-labelled antibodies.

In some embodiments of any of the methods described herein, before step (b), the method further comprises incubating the blood sample with an antigen specific for an infection for 6 hours to 3 days. In some embodiments of any of the methods described herein, the blood sample is incubated with the antigen for about 36 hours to about 60 hours.

In some embodiments of any of the methods described herein, the viable blast-transformed mononuclear cells are viable blast transformed T cells and/or viable blast transformed B cells. In some embodiments of any of the methods described herein, the viable large granular lymphocytes are NK cells.

In some embodiments of any of the methods described herein, the blood sample is a venous blood sample. In some embodiments of any of the methods described herein, the blood sample is a capillary blood sample. In some embodiments of any of the methods described herein, the capillary blood sample is a finger stick blood sample.

Some embodiments of any of the methods described herein further include generating a frozen blood sample from a portion of the blood sample. In some embodiments of any of the methods described herein, the blood sample is frozen in a solution comprising DMSO. In some embodiments of any of the methods described herein, the method comprises storing the frozen blood sample at a temperature of less than 0° C. for at least 1 day. In some embodiments of any of the methods described herein, the frozen blood sample is stored at a temperature of less than −80° C. for at least 1 day. In some embodiments of any of the methods described herein, the frozen blood sample is stored at a temperature of less than −190° C. for at least 1 day.

Some embodiments of any of the methods described herein further include thawing the frozen blood sample and performing additional immunological testing/analysis on the thawed blood sample.

In some embodiments of any of the methods described herein, the subject is identified as having an improved prognosis of cancer or infection, and a low frequency of cancer or infection screening is selected for the subject. In some embodiments of any of the methods described herein, the subject is identified as having a poor prognosis of cancer or infection, and a high frequency of cancer or infection screening is selected for the subject.

Also provided herein are methods of treating a subject not identified as having cancer or infection that include: (a) contacting a blood sample obtained from the subject with a cell viability dye; (b) after step (a), generating a microscopic slide using the blood sample; (c) identifying a number of viable blast-transformed mononuclear cells and/or a number of viable large granular lymphocytes in the microscopic slide using microscopic analysis; (d) identifying a subject having an increased number of viable blast-transformed mononuclear cells and/or an increased number of viable large granular lymphocytes, as compared to a reference level, as having an improved prognosis of cancer or infection; or identifying a subject having a decreased number of viable blast-transformed mononuclear cells and/or a decreased number of viable large granular lymphocytes, as compared to a reference level, as having a poor prognosis of cancer or infection; and (e) performing low frequency cancer or infection screening on a subject identified as having an improved prognosis of cancer or infection, or performing high frequency cancer or infection screening on a subject identified as having a poor prognosis of cancer or infection.

In some embodiments of any of the methods described herein, step (b) comprises one or more centrifugation steps. In some embodiments of any of the methods described herein, one of the centrifugation steps comprises the use of a Cytospin instrument. In some embodiments of any of the methods described herein, the microscopic slide is a permanent microscopic slide comprising a cover slip.

In some embodiments of any of the methods described herein, step (c) comprises identifying the number of viable blast-transformed mononuclear cells in the microscopic slide. In some embodiments of any of the methods described herein, step (c) comprises identifying the number of viable large granular lymphocytes in the microscopic slide. In some embodiments of any of the methods described herein, step (c) comprises identifying the number of viable blast-transformed mononuclear cell and the number of viable large granular lymphocytes in the microscopic slide.

In some embodiments of any of the methods described herein, the reference level is a 75th percentile of the median level of the number viable blast-transformed mononuclear cells or the 75th percentile of the median level of the number of viable large granular lymphocytes in a healthy patient population. In some embodiments of any of the methods described herein, the reference level is a 80th percentile of the median level of the number viable blast-transformed mononuclear cells or the 80th percentile of the median level of the number of viable large granular lymphocytes in a healthy patient population. In some embodiments of any of the methods described herein, the reference level is a 90th percentile of the median level of the number viable blast-transformed mononuclear cells or the 90th percentile of the median level of the number of viable large granular lymphocytes in a healthy patient population.

In some embodiments of any of the methods described herein, step (b) comprises the use of one or more fluorophore-labelled antibodies specific for one or more cell surface proteins. In some embodiments of any of the methods described herein, the one or more labelled antibodies bind specifically to an antigen selected from the group consisting of: CD3, CD4, CD5, CD8, CD11 c, CD14, CD16, CD19, CD20, CD25, CD27, CD28, CD38, CD39, CD45, CD45RA, CD45RO, CD56, CD57, CD62L, CD66b, CD94, CD103, CD122, CD123, CD127, CD161, CD223, CD294, CCR4, CCR6, CCR7, CXCR3, CXCR5, HLA-DR, IgA, IgD, IgE, IgG, IgM, TCRγδ, KIR, NKG2A, NKGD, NKp30, NKp44, NKp46, NKp80, CTLA-4, and GITR. In some embodiments of any of the methods described herein, the one or more labelled antibodies are one or more fluorophore-labelled antibodies.

In some embodiments of any of the methods described herein, before step (b), the method further comprises incubating the blood sample with an antigen specific for an infection for 6 hours to 3 days. In some embodiments of any of the methods described herein, the blood sample is incubated with the antigen for about 36 hours to about 60 hours.

In some embodiments of any of the methods described herein, the viable blast-transformed mononuclear cells are viable blast transformed T cells and/or viable blast transformed B cells. In some embodiments of any of the methods described herein, the viable large granular lymphocytes are NK cells.

In some embodiments of any of the methods described herein, the blood sample is a venous blood sample. In some embodiments of any of the methods described herein, the blood sample is a capillary blood sample. In some embodiments of any of the methods described herein, the capillary blood sample is a finger stick blood sample.

Some embodiments of any of the methods described herein further include generating a frozen blood sample from a portion of the blood sample. In some embodiments of any of the methods described herein, the blood sample is frozen in a solution comprising DMSO. In some embodiments of any of the methods described herein, the method comprises storing the frozen blood sample at a temperature of less than 0° C. for at least 1 day. In some embodiments of any of the methods described herein, the frozen blood sample is stored at a temperature of less than −80° C. for at least 1 day. In some embodiments of any of the methods described herein, the frozen blood sample is stored at a temperature of less than −190° C. for at least 1 day.

Some embodiments of any of the methods described herein further include thawing the frozen blood sample and performing additional immunological testing/analysis on the thawed blood sample.

In some embodiments of any of the methods described herein, the method comprises performing low frequency cancer or infection screening on a subject identified as having an improved prognosis of cancer or infection.

In some embodiments of any of the methods described herein, the method comprises performing high frequency cancer or infection screening on a subject identified as having a poor prognosis of cancer or infection.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is an exemplary image of a blast-transformed mononuclear cell.

FIG. 2 is an exemplary image of a large granular lymphocyte.

FIG. 3 is an exemplary image of a lymphocyte.

FIG. 4 is an exemplary image of unstimulated lymphocytes.

FIG. 5 is an exemplary image of stimulated lymphocytes.

### DETAILED DESCRIPTION

Described herein are methods for determining prognosis of cancer and/or infection in a subject. The method comprises identifying the number of viable blast-transformed mononuclear cells and/or the viable large granular lymphocytes in a blood sample obtained from the subject.

### Blast-Transformed Mononuclear Cells

Mononuclear cells refer to blood cells that have a single, round nucleus. When isolated from circulating blood, they are called peripheral blood mononuclear cells (PBMC), but other sources exist, such as the umbilical cord, spleen, and bone marrow.

For example, human PBMCs can be isolated from peripheral blood and identified as any blood cell with a round nucleus. PBMCs can include lymphocytes, monocytes, natural killer cells (NK cells), and dendritic cells. The cell fraction corresponding to red blood cells and granulocytes (neutrophils, basophils and eosinophils) can be removed from whole blood by density gradient centrifugation (e.g., by using the polysaccharide, Ficoll). A gradient medium with a density of 1.077 g/mL can separate whole blood into two fractions; PBMCs makes up the population of cells that remain in the low density fraction (upper fraction), whilst red blood cells and polymorphonuclear leukocytes (PMNs) (including neutrophils, eosinophils, basophils, and mast cells) have a higher density and are found in the lower fraction. Following spinning, PBMCs collect in a layer called the buffy coat, which comprise about 1% of the total sample volume. From this fraction, more specific cell types can be further isolated by purification methods that target specific cell surface proteins, such as CD4 for T helper cells, CD8 for cytotoxic T cells, CD19 for B cells, CD14/CD16 for monocytes, among others. Mononuclear cells have been described in more detail in the art, for example, by Kleiveland (“Peripheral blood mononuclear cells,” in The Impact of Food Bioactives on Health, Eds. K. Verhoeckx, P. Cotter, I. López-Exposito, C. Kleiveland, T. Lea, A. Mackie, T. Requena, D. Swiatecka, H. Wichers (Springer), 161-167 (2015)); and Sen et al. (“Perspectives on Systems Modeling of Human Peripheral Blood Mononuclear Cells,” Front Mol. Biosci. 4:96 (2018)).

Blast transformation refers to morphologic alteration of mononuclear cells, such as lymphocytes (e.g., B lymphocytes or T lymphocytes), in culture, into large blast-like cells that are able to synthesize DNA and RNA and divide mitotically. Blast transformation can be induced by interleukins, mitogens (such as phytohemagglutinins), and by specific antigens. In vivo, blast transformation of mononuclear cells can be in response to infection and/or neoplastic conditions, or in graft rejection. Blast transformation of lymphocytes by phytohemagglutinin (a non-specific mitogenic lectin) shows the same type of intra-cellular changes and subsequent development into lymphoblasts as those that occur in vivo to primed lymphocytes when they are reintroduced to the priming antigen.

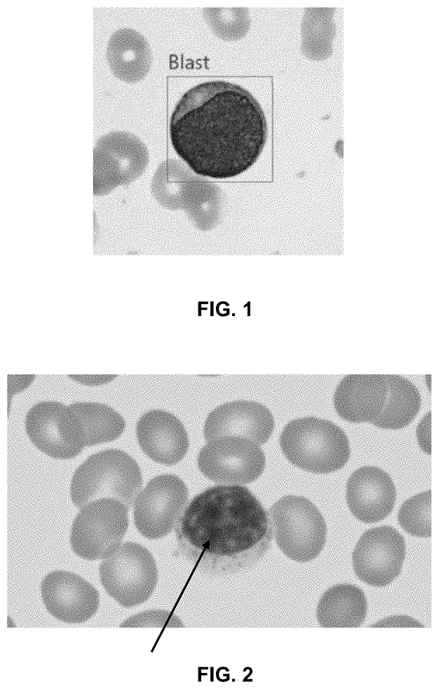

As used herein, a blast-transformed mononuclear cell refers to a lymphocyte with altered cell morphology. In certain instances, a blast-transformed mononuclear cell has a larger nucleus compared to a mononuclear cell that has not been blast-transformed. For example, a blast-transformed mononuclear cell can have a nucleus that is at least about 10% larger (e.g., at least about 20% larger, at least about 30% larger, at least about 40% larger, at least about 50% larger, at least about 60% larger, at least about 70% larger, at least about 80% larger, at least about 90% larger, or at least about 100% larger) than the nucleus of a mononuclear cell that has not been blast-transformed. Additionally, or in the alternative, the nuclear membrane of a blast-transformed mononuclear cell can be more irregular compared to the nuclear membrane of a mononuclear cell that has not been blast-transformed. An exemplary image of a blast-transformed mononuclear cell is shown in FIG. 1. A blast-transformed mononuclear cell may be formed by blast transformation of a lymphocyte (e.g., B lymphocyte or T lymphocyte), e.g., when the lymphocyte is activated by an antigen and increased in volume by nucleus and cytoplasm growth as well as new mRNA and protein synthesis. The blast-transformed mononuclear cell can then start dividing two to four times every 24 hours for three to five days, with a single blast-transformed mononuclear cell making approximately 1000 clones of its original naive lymphocyte, with each clone sharing the originally unique antigen specificity. Finally the dividing cells can differentiate into effector cells, known as plasma cells (for B cells), cytotoxic T cells, and helper T cells. See, e.g., Janeway's Immunobiology, 9th edition, Chapter 1, page 23. A blast-transformed mononuclear cell can have a size between about 10 μm to about 20 μm, Compared to a myeloblast, a blast-transformed mononuclear cell can have a less distinct nucleoli, more condensed chromatin, and/or an absence of cytoplasmic granules.

A subject having an increased number or level (e.g., at least a 1% increase, at least a 5% increase, at least a 10% increase, at least a 15% increase, at least a 20% increase, at least a 25% increase, at least a 30% increase, at least a 35% increase, at least a 40% increase, at least a 45% increase, at least a 50% increase, at least a 55% increase, at least a 60% increase, at least a 65% increase, at least a 70% increase, at least a 75% increase, at least a 80% increase, at least 85% increase, at least a 90% increase, at least a 95% increase, at least a 100% increase, at least a 120% increase, at least a 140% increase, at least a 160% increase, at least a 180% increase, at least a 200% increase, at least a 220% increase, at least a 240% increase, at least a 260% increase, at least a 280% increase, at least a 300% increase, at least a 320% increase, at least a 340% increase, at least a 360% increase, at least a 380% increase, or at least a 400% increase, or about a 1% increase to about a 400% increase, about a 1% increase to about a 380% increase, about a 1% increase to about a 360% increase, about a 1% increase to about a 340% increase, about a 1% increase to about a 320% increase, about a 1% increase to about a 300% increase, about a 1% increase to about a 280% increase, about a 1% increase to about a 260% increase, about a 1% increase to about a 240% increase, about a 1% increase to about a 220% increase, about a 1% increase to about a 200% increase, about a 1% increase to about a 180% increase, about a 1% increase to about a 160% increase, about a 1% increase to about a 140% increase, about a 1% increase to about a 120% increase, about a 1% increase to about a 100% increase, about a 1% increase to about a 90% increase, about a 1% increase to about a 80% increase, about a 1% increase to about a 70% increase, about a 1% increase to about a 60% increase, about a 1% increase to about a 50% increase, about a 1% increase to about a 40% increase, about a 1% increase to about a 30% increase, about a 1% increase to about a 25% increase, about a 1% increase to about a 20% increase, about a 1% increase to about a 15% increase, about a 1% increase to about a 10% increase, about a 1% increase to about a 5% increase, about a 5% increase to about a 400% increase, about a 5% increase to about a 380% increase, about a 5% increase to about a 360% increase, about a 5% increase to about a 340% increase, about a 5% increase to about a 320% increase, about a 5% increase to about a 300% increase, about a 5% increase to about a 280% increase, about a 5% increase to about a 260% increase, about a 5% increase to about a 240% increase, about a 5% increase to about a 220% increase, about a 5% increase to about a 200% increase, about a 5% increase to about a 180% increase, about a 5% increase to about a 160% increase, about a 5% increase to about a 140% increase, about a 5% increase to about a 120% increase, about a 5% increase to about a 100% increase, about a 5% increase to about a 90% increase, about a 5% increase to about a 80% increase, about a 5% increase to about a 70% increase, about a 5% increase to about a 60% increase, about a 5% increase to about a 50% increase, about a 5% increase to about a 40% increase, about a 5% increase to about a 30% increase, about a 5% increase to about a 25% increase, about a 5% increase to about a 20% increase, about a 5% increase to about a 15% increase, about a 5% increase to about a 10% increase, about a 10% increase to about a 400% increase, about a 10% increase to about a 380% increase, about a 10% increase to about a 360% increase, about a 10% increase to about a 340% increase, about a 10% increase to about a 320% increase, about a 10% increase to about a 300% increase, about a 10% increase to about a 280% increase, about a 10% increase to about a 260% increase, about a 10% increase to about a 240% increase, about a 10% increase to about a 220% increase, about a 10% increase to about a 200% increase, about a 10% increase to about a 180% increase, about a 10% increase to about a 160% increase, about a 10% increase to about a 140% increase, about a 10% increase to about a 120% increase, about a 10% increase to about a 100% increase, about a 10% increase to about a 90% increase, about a 10% increase to about a 80% increase, about a 10% increase to about a 70% increase, about a 10% increase to about a 60% increase, about a 10% increase to about a 50% increase, about a 10% increase to about a 40% increase, about a 10% increase to about a 30% increase, about a 10% increase to about a 25% increase, about a 10% increase to about a 20% increase, about a 10% increase to about a 15% increase, about a 15% increase to about a 400% increase, about a 15% increase to about a 380% increase, about a 15% increase to about a 360% increase, about a 15% increase to about a 340% increase, about a 15% increase to about a 320% increase, about a 15% increase to about a 300% increase, about a 15% increase to about a 280% increase, about a 15% increase to about a 260% increase, about a 15% increase to about a 240% increase, about a 15% increase to about a 220% increase, about a 15% increase to about a 200% increase, about a 15% increase to about a 180% increase, about a 15% increase to about a 160% increase, about a 15% increase to about a 140% increase, about a 15% increase to about a 120% increase, about a 15% increase to about a 100% increase, about a 15% increase to about a 90% increase, about a 15% increase to about a 80% increase, about a 15% increase to about a 70% increase, about a 15% increase to about a 60% increase, about a 15% increase to about a 50% increase, about a 15% increase to about a 40% increase, about a 15% increase to about a 30% increase, about a 15% increase to about a 25% increase, about a 15% increase to about a 20% increase, about a 20% increase to about a 400% increase, about a 20% increase to about a 380% increase, about a 20% increase to about a 360% increase, about a 20% increase to about a 340% increase, about a 20% increase to about a 320% increase, about a 20% increase to about a 300% increase, about a 20% increase to about a 280% increase, about a 20% increase to about a 260% increase, about a 20% increase to about a 240% increase, about a 20% increase to about a 220% increase, about a 20% increase to about a 200% increase, about a 20% increase to about a 180% increase, about a 20% increase to about a 160% increase, about a 20% increase to about a 140% increase, about a 20% increase to about a 120% increase, about a 20% increase to about a 100% increase, about a 20% increase to about a 90% increase, about a 20% increase to about a 80% increase, about a 20% increase to about a 70% increase, about a 20% increase to about a 60% increase, about a 20% increase to about a 50% increase, about a 20% increase to about a 40% increase, about a 20% increase to about a 30% increase, about a 20% increase to about a 25% increase, about a 25% increase to about a 400% increase, about a 25% increase to about a 380% increase, about a 25% increase to about a 360% increase, about a 25% increase to about a 340% increase, about a 25% increase to about a 320% increase, about a 25% increase to about a 300% increase, about a 25% increase to about a 280% increase, about a 25% increase to about a 260% increase, about a 25% increase to about a 240% increase, about a 25% increase to about a 220% increase, about a 25% increase to about a 200% increase, about a 25% increase to about a 180% increase, about a 25% increase to about a 160% increase, about a 25% increase to about a 140% increase, about a 25% increase to about a 120% increase, about a 25% increase to about a 100% increase, about a 25% increase to about a 90% increase, about a 25% increase to about a 80% increase, about a 25% increase to about a 70% increase, about a 25% increase to about a 60% increase, about a 25% increase to about a 50% increase, about a 25% increase to about a 40% increase, about a 25% increase to about a 30% increase, about a 30% increase to about a 400% increase, about a 30% increase to about a 380% increase, about a 30% increase to about a 360% increase, about a 30% increase to about a 340% increase, about a 30% increase to about a 320% increase, about a 30% increase to about a 300% increase, about a 30% increase to about a 280% increase, about a 30% increase to about a 260% increase, about a 30% increase to about a 240% increase, about a 30% increase to about a 220% increase, about a 30% increase to about a 200% increase, about a 30% increase to about a 180% increase, about a 30% increase to about a 160% increase, about a 30% increase to about a 140% increase, about a 30% increase to about a 120% increase, about a 30% increase to about a 100% increase, about a 30% increase to about a 90% increase, about a 30% increase to about a 80% increase, about a 30% increase to about a 70% increase, about a 30% increase to about a 60% increase, about a 30% increase to about a 50% increase, about a 30% increase to about a 40% increase, about a 40% increase to about a 400% increase, about a 40% increase to about a 380% increase, about a 40% increase to about a 360% increase, about a 40% increase to about a 340% increase, about a 40% increase to about a 320% increase, about a 40% increase to about a 300% increase, about a 40% increase to about a 280% increase, about a 40% increase to about a 260% increase, about a 40% increase to about a 240% increase, about a 40% increase to about a 220% increase, about a 40% increase to about a 200% increase, about a 40% increase to about a 180% increase, about a 40% increase to about a 160% increase, about a 40% increase to about a 140% increase, about a 40% increase to about a 120% increase, about a 40% increase to about a 100% increase, about a 40% increase to about a 90% increase, about a 40% increase to about a 80% increase, about a 40% increase to about a 70% increase, about a 40% increase to about a 60% increase, about a 40% increase to about a 50% increase, about a 50% increase to about a 400% increase, about a 50% increase to about a 380% increase, about a 50% increase to about a 360% increase, about a 50% increase to about a 340% increase, about a 50% increase to about a 320% increase, about a 50% increase to about a 300% increase, about a 50% increase to about a 280% increase, about a 50% increase to about a 260% increase, about a 50% increase to about a 240% increase, about a 50% increase to about a 220% increase, about a 50% increase to about a 200% increase, about a 50% increase to about a 180% increase, about a 50% increase to about a 160% increase, about a 50% increase to about a 140% increase, about a 50% increase to about a 120% increase, about a 50% increase to about a 100% increase, about a 50% increase to about a 90% increase, about a 50% increase to about a 80% increase, about a 50% increase to about a 70% increase, about a 50% increase to about a 60% increase, about a 60% increase to about a 400% increase, about a 60% increase to about a 380% increase, about a 60% increase to about a 360% increase, about a 60% increase to about a 340% increase, about a 60% increase to about a 320% increase, about a 60% increase to about a 300% increase, about a 60% increase to about a 280% increase, about a 60% increase to about a 260% increase, about a 60% increase to about a 240% increase, about a 60% increase to about a 220% increase, about a 60% increase to about a 200% increase, about a 60% increase to about a 180% increase, about a 60% increase to about a 160% increase, about a 60% increase to about a 140% increase, about a 60% increase to about a 120% increase, about a 60% increase to about a 100% increase, about a 60% increase to about a 90% increase, about a 60% increase to about a 80% increase, about a 60% increase to about a 70% increase, about a 70% increase to about a 400% increase, about a 70% increase to about a 380% increase, about a 70% increase to about a 360% increase, about a 70% increase to about a 340% increase, about a 70% increase to about a 320% increase, about a 70% increase to about a 300% increase, about a 70% increase to about a 280% increase, about a 70% increase to about a 260% increase, about a 70% increase to about a 240% increase, about a 70% increase to about a 220% increase, about a 70% increase to about a 200% increase, about a 70% increase to about a 180% increase, about a 70% increase to about a 160% increase, about a 70% increase to about a 140% increase, about a 70% increase to about a 120% increase, about a 70% increase to about a 100% increase, about a 70% increase to about a 90% increase, about a 70% increase to about a 80% increase, about a 80% increase to about a 400% increase, about a 80% increase to about a 380% increase, about a 80% increase to about a 360% increase, about a 80% increase to about a 340% increase, about a 80% increase to about a 320% increase, about a 80% increase to about a 300% increase, about a 80% increase to about a 280% increase, about a 80% increase to about a 260% increase, about a 80% increase to about a 240% increase, about a 80% increase to about a 220% increase, about a 80% increase to about a 200% increase, about a 80% increase to about a 180% increase, about a 80% increase to about a 160% increase, about a 80% increase to about a 140% increase, about a 80% increase to about a 120% increase, about a 80% increase to about a 100% increase, about a 80% increase to about a 90% increase, about a 90% increase to about a 400% increase, about a 90% increase to about a 380% increase, about a 90% increase to about a 360% increase, about a 90% increase to about a 340% increase, about a 90% increase to about a 320% increase, about a 90% increase to about a 300% increase, about a 90% increase to about a 280% increase, about a 90% increase to about a 260% increase, about a 90% increase to about a 240% increase, about a 90% increase to about a 220% increase, about a 90% increase to about a 200% increase, about a 90% increase to about a 180% increase, about a 90% increase to about a 160% increase, about a 90% increase to about a 140% increase, about a 90% increase to about a 120% increase, about a 90% increase to about a 100% increase, about a 100% increase to about a 400% increase, about a 100% increase to about a 380% increase, about a 100% increase to about a 360% increase, about a 100% increase to about a 340% increase, about a 100% increase to about a 320% increase, about a 100% increase to about a 300% increase, about a 100% increase to about a 280% increase, about a 100% increase to about a 260% increase, about a 100% increase to about a 240% increase, about a 100% increase to about a 220% increase, about a 100% increase to about a 200% increase, about a 100% increase to about a 180% increase, about a 100% increase to about a 160% increase, about a 100% increase to about a 140% increase, about a 100% increase to about a 120% increase, about a 120% increase to about a 400% increase, about a 120% increase to about a 380% increase, about a 120% increase to about a 360% increase, about a 120% increase to about a 340% increase, about a 120% increase to about a 320% increase, about a 120% increase to about a 300% increase, about a 120% increase to about a 280% increase, about a 120% increase to about a 260% increase, about a 120% increase to about a 240% increase, about a 120% increase to about a 220% increase, about a 120% increase to about a 200% increase, about a 120% increase to about a 180% increase, about a 120% increase to about a 160% increase, about a 120% increase to about a 140% increase, about a 140% increase to about a 400% increase, about a 140% increase to about a 380% increase, about a 140% increase to about a 360% increase, about a 140% increase to about a 340% increase, about a 140% increase to about a 320% increase, about a 140% increase to about a 300% increase, about a 140% increase to about a 280% increase, about a 140% increase to about a 260% increase, about a 140% increase to about a 240% increase, about a 140% increase to about a 220% increase, about a 140% increase to about a 200% increase, about a 140% increase to about a 180% increase, about a 140% increase to about a 160% increase, about a 160% increase to about a 400% increase, about a 160% increase to about a 380% increase, about a 160% increase to about a 360% increase, about a 160% increase to about a 340% increase, about a 160% increase to about a 320% increase, about a 160% increase to about a 300% increase, about a 160% increase to about a 280% increase, about a 160% increase to about a 260% increase, about a 160% increase to about a 240% increase, about a 160% increase to about a 220% increase, about a 160% increase to about a 200% increase, about a 160% increase to about a 180% increase, about a 180% increase to about a 400% increase, about a 180% increase to about a 380% increase, about a 180% increase to about a 360% increase, about a 180% increase to about a 340% increase, about a 180% increase to about a 320% increase, about a 180% increase to about a 300% increase, about a 180% increase to about a 280% increase, about a 180% increase to about a 260% increase, about a 180% increase to about a 240% increase, about a 180% increase to about a 220% increase, about a 180% increase to about a 200% increase, about a 200% increase to about a 400% increase, about a 200% increase to about a 380% increase, about a 200% increase to about a 360% increase, about a 200% increase to about a 340% increase, about a 200% increase to about a 320% increase, about a 200% increase to about a 300% increase, about a 200% increase to about a 280% increase, about a 200% increase to about a 260% increase, about a 200% increase to about a 240% increase, about a 200% increase to about a 220% increase, about a 220% increase to about a 400% increase, about a 220% increase to about a 380% increase, about a 220% increase to about a 360% increase, about a 220% increase to about a 340% increase, about a 220% increase to about a 320% increase, about a 220% increase to about a 300% increase, about a 220% increase to about a 280% increase, about a 220% increase to about a 260% increase, about a 220% increase to about a 240% increase, about a 240% increase to about a 400% increase, about a 240% increase to about a 380% increase, about a 240% increase to about a 360% increase, about a 240% increase to about a 340% increase, about a 240% increase to about a 320% increase, about a 240% increase to about a 300% increase, about a 240% increase to about a 280% increase, about a 240% increase to about a 260% increase, about a 260% increase to about a 400% increase, about a 260% increase to about a 380% increase, about a 260% increase to about a 360% increase, about a 260% increase to about a 340% increase, about a 260% increase to about a 320% increase, about a 260% increase to about a 300% increase, about a 260% increase to about a 280% increase, about a 280% increase to about a 400% increase, about a 280% increase to about a 380% increase, about a 280% increase to about a 360% increase, about a 280% increase to about a 340% increase, about a 280% increase to about a 320% increase, about a 280% increase to about a 300% increase, about a 300% increase to about a 400% increase, about a 300% increase to about a 380% increase, about a 300% increase to about a 360% increase, about a 300% increase to about a 340% increase, about a 300% increase to about a 320% increase, about a 320% increase to about a 400% increase, about a 320% increase to about a 380% increase, about a 320% increase to about a 360% increase, about a 320% increase to about a 340% increase, about a 340% increase to about a 400% increase, about a 340% increase to about a 380% increase, about a 340% increase to about a 360% increase, about a 360% increase to about a 400% increase, about a 360% increase to about a 380% increase, or about a 380% increase to about a 400% increase) of viable blast-transformed mononuclear cells, as compared to a reference level (e.g., any of the exemplary reference levels described herein), may be identified as having improved prognosis of cancer and/or infection. For example, a subject having an increased number of viable blast-transformed mononuclear cells as compared to a reference level (e.g., any of the exemplary reference levels described herein), may be identified as having improved prognosis of cancer or infection.

In the alternative, a subject having a decreased number or level (e.g., at least a 1% decrease, at least a 5% decrease, at least a 10% decrease, at least a 15% decrease, at least a 20% decrease, at least a 25% decrease, at least a 30% decrease, at least a 35% decrease, at least a 40% decrease, at least a 45% decrease, at least a 50% decrease, at least a 55% decrease, at least a 60% decrease, at least a 65% decrease, at least 70% decrease, at least a 75% decrease, at least a 80% decrease, at least a 85% decrease, at least a 90% decrease, at least a 95% decrease, or at least a 99% decrease, or about a 1% decrease to about a 99% decrease, about a 1% decrease to about a 95% decrease, about a 1% decrease to about a 95% decrease, about a 1% decrease to about a 90% decrease, about a 1% decrease to about a 85% decrease, about a 1% decrease to about a 80% decrease, about a 1% decrease to about a 75% decrease, about a 1% decrease to about a 70% decrease, about a 1% decrease to about a 65% decrease, about a 1% decrease to about a 60% decrease, about a 1% decrease to about a 55% decrease, about a 1% decrease to about a 50% decrease, about a 1% decrease to about a 45% decrease, about a 1% decrease to about a 40% decrease, about a 1% decrease to about a 35% decrease, about a 1% decrease to about a 30% decrease, about a 1% decrease to about a 25% decrease, about a 1% decrease to about a 20% decrease, about a 1% decrease to about a 15% decrease, about a 1% decrease to about a 10% decrease, about a 1% decrease to about a 5% decrease, about a 5% decrease to about a 99% decrease, about a 5% decrease to about a 95% decrease, about a 5% decrease to about a 95% decrease, about a 5% decrease to about a 90% decrease, about a 5% decrease to about a 85% decrease, about a 5% decrease to about a 80% decrease, about a 5% decrease to about a 75% decrease, about a 5% decrease to about a 70% decrease, about a 5% decrease to about a 65% decrease, about a 5% decrease to about a 60% decrease, about a 5% decrease to about a 55% decrease, about a 5% decrease to about a 50% decrease, about a 5% decrease to about a 45% decrease, about a 5% decrease to about a 40% decrease, about a 5% decrease to about a 35% decrease, about a 5% decrease to about a 30% decrease, about a 5% decrease to about a 25% decrease, about a 5% decrease to about a 20% decrease, about a 5% decrease to about a 15% decrease, about a 5% decrease to about a 10% decrease, about a 10% decrease to about a 99% decrease, about a 10% decrease to about a 95% decrease, about a 10% decrease to about a 95% decrease, about a 10% decrease to about a 90% decrease, about a 10% decrease to about a 85% decrease, about a 10% decrease to about a 80% decrease, about a 10% decrease to about a 75% decrease, about a 10% decrease to about a 70% decrease, about a 10% decrease to about a 65% decrease, about a 10% decrease to about a 60% decrease, about a 10% decrease to about a 55% decrease, about a 10% decrease to about a 50% decrease, about a 10% decrease to about a 45% decrease, about a 10% decrease to about a 40% decrease, about a 10% decrease to about a 35% decrease, about a 10% decrease to about a 30% decrease, about a 10% decrease to about a 25% decrease, about a 10% decrease to about a 20% decrease, about a 10% decrease to about a 15% decrease, about a 15% decrease to about a 99% decrease, about a 15% decrease to about a 95% decrease, about a 15% decrease to about a 95% decrease, about a 15% decrease to about a 90% decrease, about a 15% decrease to about a 85% decrease, about a 15% decrease to about a 80% decrease, about a 15% decrease to about a 75% decrease, about a 15% decrease to about a 70% decrease, about a 15% decrease to about a 65% decrease, about a 15% decrease to about a 60% decrease, about a 15% decrease to about a 55% decrease, about a 15% decrease to about a 50% decrease, about a 15% decrease to about a 45% decrease, about a 15% decrease to about a 40% decrease, about a 15% decrease to about a 35% decrease, about a 15% decrease to about a 30% decrease, about a 15% decrease to about a 25% decrease, about a 15% decrease to about a 20% decrease, about a 20% decrease to about a 99% decrease, about a 20% decrease to about a 95% decrease, about a 20% decrease to about a 95% decrease, about a 20% decrease to about a 90% decrease, about a 20% decrease to about a 85% decrease, about a 20% decrease to about a 80% decrease, about a 20% decrease to about a 75% decrease, about a 20% decrease to about a 70% decrease, about a 20% decrease to about a 65% decrease, about a 20% decrease to about a 60% decrease, about a 20% decrease to about a 55% decrease, about a 20% decrease to about a 50% decrease, about a 20% decrease to about a 45% decrease, about a 20% decrease to about a 40% decrease, about a 20% decrease to about a 35% decrease, about a 20% decrease to about a 30% decrease, about a 20% decrease to about a 25% decrease, about a 25% decrease to about a 99% decrease, about a 25% decrease to about a 95% decrease, about a 25% decrease to about a 95% decrease, about a 25% decrease to about a 90% decrease, about a 25% decrease to about a 85% decrease, about a 25% decrease to about a 80% decrease, about a 25% decrease to about a 75% decrease, about a 25% decrease to about a 70% decrease, about a 25% decrease to about a 65% decrease, about a 25% decrease to about a 60% decrease, about a 25% decrease to about a 55% decrease, about a 25% decrease to about a 50% decrease, about a 25% decrease to about a 45% decrease, about a 25% decrease to about a 40% decrease, about a 25% decrease to about a 35% decrease, about a 25% decrease to about a 30% decrease, about a 30% decrease to about a 99% decrease, about a 30% decrease to about a 95% decrease, about a 30% decrease to about a 95% decrease, about a 30% decrease to about a 90% decrease, about a 30% decrease to about a 85% decrease, about a 30% decrease to about a 80% decrease, about a 30% decrease to about a 75% decrease, about a 30% decrease to about a 70% decrease, about a 30% decrease to about a 65% decrease, about a 30% decrease to about a 60% decrease, about a 30% decrease to about a 55% decrease, about a 30% decrease to about a 50% decrease, about a 30% decrease to about a 45% decrease, about a 30% decrease to about a 40% decrease, about a 30% decrease to about a 35% decrease, about a 35% decrease to about a 99% decrease, about a 35% decrease to about a 95% decrease, about a 35% decrease to about a 95% decrease, about a 35% decrease to about a 90% decrease, about a 35% decrease to about a 85% decrease, about a 35% decrease to about a 80% decrease, about a 35% decrease to about a 75% decrease, about a 35% decrease to about a 70% decrease, about a 35% decrease to about a 65% decrease, about a 35% decrease to about a 60% decrease, about a 35% decrease to about a 55% decrease, about a 35% decrease to about a 50% decrease, about a 35% decrease to about a 45% decrease, about a 35% decrease to about a 40% decrease, about a 40% decrease to about a 99% decrease, about a 40% decrease to about a 95% decrease, about a 40% decrease to about a 95% decrease, about a 40% decrease to about a 90% decrease, about a 40% decrease to about a 85% decrease, about a 40% decrease to about a 80% decrease, about a 40% decrease to about a 75% decrease, about a 40% decrease to about a 70% decrease, about a 40% decrease to about a 65% decrease, about a 40% decrease to about a 60% decrease, about a 40% decrease to about a 55% decrease, about a 40% decrease to about a 50% decrease, about a 40% decrease to about a 45% decrease, about a 45% decrease to about a 99% decrease, about a 45% decrease to about a 95% decrease, about a 45% decrease to about a 95% decrease, about a 45% decrease to about a 90% decrease, about a 45% decrease to about a 85% decrease, about a 45% decrease to about a 80% decrease, about a 45% decrease to about a 75% decrease, about a 45% decrease to about a 70% decrease, about a 45% decrease to about a 65% decrease, about a 45% decrease to about a 60% decrease, about a 45% decrease to about a 55% decrease, about a 45% decrease to about a 50% decrease, about a 50% decrease to about a 99% decrease, about a 50% decrease to about a 95% decrease, about a 50% decrease to about a 95% decrease, about a 50% decrease to about a 90% decrease, about a 50% decrease to about a 85% decrease, about a 50% decrease to about a 80% decrease, about a 50% decrease to about a 75% decrease, about a 50% decrease to about a 70% decrease, about a 50% decrease to about a 65% decrease, about a 50% decrease to about a 60% decrease, about a 50% decrease to about a 55% decrease, about a 55% decrease to about a 99% decrease, about a 55% decrease to about a 95% decrease, about a 55% decrease to about a 95% decrease, about a 55% decrease to about a 90% decrease, about a 55% decrease to about a 85% decrease, about a 55% decrease to about a 80% decrease, about a 55% decrease to about a 75% decrease, about a 55% decrease to about a 70% decrease, about a 55% decrease to about a 65% decrease, about a 55% decrease to about a 60% decrease, about a 60% decrease to about a 99% decrease, about a 60% decrease to about a 95% decrease, about a 60% decrease to about a 95% decrease, about a 60% decrease to about a 90% decrease, about a 60% decrease to about a 85% decrease, about a 60% decrease to about a 80% decrease, about a 60% decrease to about a 75% decrease, about a 60% decrease to about a 70% decrease, about a 60% decrease to about a 65% decrease, about a 65% decrease to about a 99% decrease, about a 65% decrease to about a 95% decrease, about a 65% decrease to about a 95% decrease, about a 65% decrease to about a 90% decrease, about a 65% decrease to about a 85% decrease, about a 65% decrease to about a 80% decrease, about a 65% decrease to about a 75% decrease, about a 65% decrease to about a 70% decrease, about a 70% decrease to about a 99% decrease, about a 70% decrease to about a 95% decrease, about a 70% decrease to about a 95% decrease, about a 70% decrease to about a 90% decrease, about a 70% decrease to about a 85% decrease, about a 70% decrease to about a 80% decrease, about a 70% decrease to about a 75% decrease, about a 75% decrease to about a 99% decrease, about a 75% decrease to about a 95% decrease, about a 75% decrease to about a 95% decrease, about a 75% decrease to about a 90% decrease, about a 75% decrease to about a 85% decrease, about a 75% decrease to about a 80% decrease, about a 80% decrease to about a 99% decrease, about a 80% decrease to about a 95% decrease, about a 80% decrease to about a 95% decrease, about a 80% decrease to about a 90% decrease, about a 80% decrease to about a 85% decrease, about a 85% decrease to about a 99% decrease, about a 85% decrease to about a 95% decrease, about a 85% decrease to about a 95% decrease, about a 85% decrease to about a 90% decrease, about a 90% decrease to about a 99% decrease, about a 90% decrease to about a 95% decrease, about a 90% decrease to about a 95% decrease, about a 95% decrease to about a 99% decrease, about a 95% decrease to about a 95% decrease, or about a 95% decrease to about a 99% decrease) of viable blast-transformed mononuclear cells, as compared to a reference level (e.g., any of the exemplary reference levels described herein), may be identified as having poor prognosis of cancer and/or infection.

In certain instances, a reference level is about the 70th percentile, about the 71st percentile, about the 72nd percentile, about the 73rd percentile, about the 74th percentile, about the 75th percentile, about the 76th percentile, about the 77th percentile, about the 78th percentile, about the 79th percentile, about the 80th percentile, about the 81st percentile, about the 82nd percentile, about the 83rd percentile, about the 84th percentile, about the 85th percentile, about the 86th percentile, about the 87th percentile, about the 88th percentile, about the 89th percentile, about the 90th percentile, about the 91st percentile, about the 92nd percentile, about the 93rd percentile, about the 94th percentile, about the 95th percentile, about the 96th percentile, about the 97th percentile, about the 98th percentile, or about the 99th percentile)) of the median level of the number or level of viable blast-transformed mononuclear cells in a healthy patient population. A healthy patient population may be a patient population that is not currently diagnosed with a cancer and/or an infection, or has not been diagnosed with a cancer and/or an infection within the last 5 years (e.g., within the last 4 years, 3 years, 2 years, 1 year, 11 months, 10 months, 9 months, 8 months, 7 months, 6 months, 5 months, 4 months, 3 months, 2 months, 1 month, 3 weeks, 2 weeks, or 1 week).

### Large Granular Lymphocytes

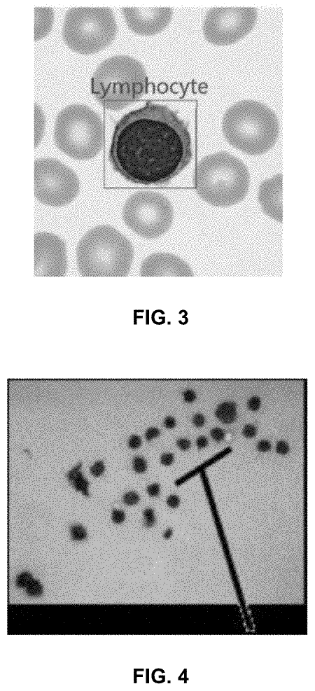

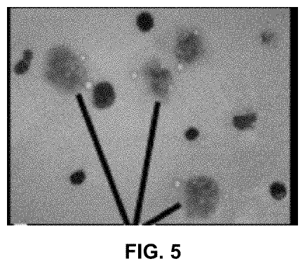

Large granular lymphocytes (LGL) are lymphoid cells that are characterized by either a T-cell or a natural killer (NK) phenotype that physiologically participate in innate immunity and immunosurveillance. Morphologically, large granular lymphocytes are more than twice the diameter of erythrocytes, and are characterized by round or reniform nuclei, mature chromatin, excessive cytoplasm, and prominent azurophilic cytoplasmic granules. Normally, large granular lymphocytes comprise 10% to 15% of blood mononuclear cells which may be either surface CD3+ (T-cell) or surface CD3⁻ (NK cell). Most normal large granular lymphocytes in the peripheral blood are NK cells, whilst some are T lymphocytes. Large granular lymphocytes have been described in more detail in the art, for example, by Fattizzo et al. (“Large Granular Lymphocyte Expansion in Myeloid Diseases and Bone Marrow Failure Syndromes: Whoever Seeks Finds,” Front. Oncol. 11:748610 (2021)); Vivier et al. (“Innate or adaptive immunity? The example of natural killer cells,” Science 331(6013):44-49 (2011)); and Oshimi (“Clinical Features, Pathogenesis, and Treatment of Large Granular Lymphocyte Leukemias,” Intern. Med. 56:1759-1769 (2017)). An exemplary image of a large granular lymphocyte is shown in FIG. 2. Exemplary images of a lymphocyte, unstimulated lymphocytes, and stimulated lymphocytes are shown in FIGS. 3-5, respectively.

In some embodiments, the large granular lymphocytes are NK cells. NK cells provide rapid responses to virus-infected cell and other intracellular pathogens acting at around 3 days after infection, and also respond to tumor formation. Typically, immune cells detect the major histocompatibility complex (MHC) presented on infected cell surfaces, triggering cytokine release, and causing the death of the infected cell by lysis or apoptosis. NK cells are unique, however, as they have the ability to recognize and kill stressed cells in the absence of antibodies and MEC, allowing for a much faster immune reaction. They were named “natural killers” because of the notion that they do not require activation to kill cells that are missing “self” markers of MHC class 1. This role is especially important because harmful cells that are missing MEC I markers cannot be detected and destroyed by other immune cells, such as T lymphocyte cells.

As described herein a subject with an increased number or level (e.g., at least a 1% increase, at least a 5% increase, at least a 10% increase, at least a 15% increase, at least a 20% increase, at least a 25% increase, at least a 30% increase, at least a 35% increase, at least a 40% increase, at least a 45% increase, at least a 50% increase, at least a 55% increase, at least a 60% increase, at least a 65% increase, at least a 70% increase, at least a 75% increase, at least a 80% increase, at least 85% increase, at least a 90% increase, at least a 95% increase, at least a 100% increase, at least a 120% increase, at least a 140% increase, at least a 160% increase, at least a 180% increase, at least a 200% increase, at least a 220% increase, at least a 240% increase, at least a 260% increase, at least a 280% increase, at least a 300% increase, at least a 320% increase, at least a 340% increase, at least a 360% increase, at least a 380% increase, or at least a 400% increase, or about a 1% increase to about a 400% increase (or any of the subranges of this range described herein)) of viable large granular lymphocytes, as compared to a reference level (e.g., any of the exemplary reference levels described herein), may be identified as having improved prognosis of one or more cancers and/or infections.

In other examples, a subject with a decreased number or level (e.g., at least a 1% decrease, at least a 5% decrease, at least a 10% decrease, at least a 15% decrease, at least a 20% decrease, at least a 25% decrease, at least a 30% decrease, at least a 35% decrease, at least a 40% decrease, at least a 45% decrease, at least a 50% decrease, at least a 55% decrease, at least a 60% decrease, at least a 65% decrease, at least 70% decrease, at least a 75% decrease, at least a 80% decrease, at least a 85% decrease, at least a 90% decrease, at least a 95% decrease, or at least a 99% decrease, or about a 1% decrease to about a 99% decrease (or any of the subranges of this range described herein)), of viable large granular lymphocytes, as compared to a reference level (e.g., any of the exemplary reference levels described herein), may be identified as having poor prognosis of cancer and/or infection.

In certain instances, a reference level is about the 70th percentile, about the 71st percentile, about the 72nd percentile, about the 73rd percentile, about the 74th percentile, about the 75th percentile, about the 76th percentile, about the 77th percentile, about the 78th percentile, about the 79th percentile, about the 80th percentile, about the 81st percentile, about the 82nd percentile, about the 83rd percentile, about the 84th percentile, about the 85th percentile, about the 86th percentile, about the 87th percentile, about the 88th percentile, about the 89th percentile, about the 90th percentile, about the 91st percentile, about the 92nd percentile, about the 93rd percentile, about the 94th percentile, about the 95th percentile, about the 96th percentile, about the 97th percentile, about the 98th percentile, or about the 99th percentile)) of the median level of the number or level of viable large granular lymphocytes in a healthy patient population. A healthy patient population may be a patient population that is not currently diagnosed with a cancer and/or an infection, or has not been diagnosed with a cancer and/or an infection within the last 5 years (e.g., within the last 4 years, 3 years, 2 years, 1 year, 11 months, 10 months, 9 months, 8 months, 7 months, 6 months, 5 months, 4 months, 3 months, 2 months, 1 month, 3 weeks, 2 weeks, or 1 week).

### Sample Preparation

The present disclosure provides methods for assessing a subject's prognosis of cancer and/or infection by analyzing blood sample(s) from the subject. Described herein are methods for collection and preparation of blood sample(s) for use in the present methods.

### Collection of Blood Sample

Blood sample(s) can be collected from a subject by standard phlebotomy methods known in the art (see, e.g., WHO Guidelines on Drawing Blood: Best Practices in Phlebotomy. Geneva: World Health Organization; 2010. 2, Best practices in phlebotomy). In certain instances, blood sample(s) can be collected with an anticoagulant, such as heparin, ethylenediaminetetraacetic acid (EDTA), citrate, acid citrate dextrose (ACD), or citrate phosphate dextrose (CPD). A blood sample for use in the present methods can be a venous blood sample. A venous blood sample can be collected from a subject by venipuncture. Additionally, or in the alternative, a blood sample for use in the present methods can be a capillary blood sample. A capillary blood sample can be a finger stick blood sample (e.g., collected from a subject by finger stick procedure, such as by pricking a finger) or a heel stick blood sample (e.g., collected from a subject by heel stick procedure, such as by pricking a heel).

Although it is generally preferable to prepare cells for analysis immediately after collection of blood samples, in some instances (e.g., shipping specimens from a distant site), it may be necessary to store blood sample(s) for brief periods of time prior to further processing (e.g., prior to PBMC isolation and analysis). For example, blood sample(s) can be stored for at least 12 hours (e.g., at least 12-18 hours, 18-24 hours, 1-2 days, 1-3 days, 1-4 days, 1-5 days, 1-6 days, 1-7 days, 1-2 weeks, 1-3 weeks, 1-4 weeks, 1-2 months, 1-3 months, 1-4 months, 1-5 months, 1-6 months, or more (e.g., at least 12 hours, 18 hours, 24 hours, 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, or more)). Several commercially available reagents can be used to treat blood sample(s) for storage. For example, for use in the present methods, blood sample(s) can be stored using TRANSFIX (CYTOMARK) and/or CYTO-CHEX (STRECK). Additionally, or in the alternative, blood sample(s) can be stored in dimethyl sulfoxide (DMSO).

In some instances, blood samples can be stored at room temperature. In some instances, blood samples can be stored at a temperature of about 0° C. to about 21° C. (e.g., about 1° C. to about 21° C., about 4° C. to about 21° C., about 10° C. to about 21° C., about 18° C. to about 21° C., about 0° C. to about 18° C., about 1° C. to about 18° C., about 4° C. to about 18° C., about 10° C. to about 18° C., about 0° C. to about 10° C., about 1° C. to about 10° C., or about 4° C. to about 10° C. (e.g., at about 0° C., 1° C., 4° C., 6° C., 8° C., 10° C., 12° C., 14° C., 16° C., 18° C., 20° C., or 21° C.)). In certain instances, blood samples can be frozen and stored at a temperature of less than 0° C. (e.g., less than −1° C., less than −10° C., less than −20° C., less than −30° C., less than −40° C., less than −50° C., less than −60° C., less than −70° C., less than −80° C., less than −90° C., less than −100° C., less than −110° C., less than −120° C., less than −130° C., less than −140° C., less than −150° C., less than −160° C., less than −170° C., less than −180° C., less than −190° C., or less than −200° C.). Before further processing (e.g., before isolating PBMCs from such blood sample(s) and/or preparing a microscope slide as described herein), the frozen blood sample(s) may be thawed. Each assay, for which the blood sample(s) are stored (e.g., frozen and stored), can be validated on the stored specimens, such that assay data from a fresh specimen can be compared to that from the same specimen after storage, so as to assure that similar, if not identical, data are obtained.

### Isolation of Cells

Cells can be isolated from the blood sample(s) using methods known in the art. See, e.g., Dagur and McCoy J P Jr., “Collection, Storage, and Preparation of Human Blood Cells,” Curr. Protoc. Cytom. 73:5.1.1-5.1.16 (2015).

In certain instances, PBMCs (e.g., lymphocytes, NK cells, etc.) can be isolated from the blood sample(s) by elimination of erythrocytes from the samples(s), such as by lysis. Lysis is quicker than gradient separation and in general leaves the remaining white cell populations relatively unperturbed. Furthermore, the yield of mononuclear cells from blood by lysis of erythrocytes is much higher than yields from density gradient separations. This procedure, in which erythrocytes are lysed with osmotic shock (e.g., cell membrane lysis by ammonium chloride), may be used for unstained blood or blood that has already been incubated with monoclonal antibodies. In general, this method will not affect the pattern of staining observed for most lymphoid markers. The viability of white blood cells subjected to this treatment is good. Erythrocytes can be lysed by using homemade RBC lysis buffer as well as commercial RBC lysis buffer without fixative, such as, ACK LYSIS BUFFER (Quality Biological), OPTILYSE (Beckman Coulter), PHARMLYSE (BD Bioscience), IOTEST3 (Beckman Coulter) HI-YIELD LYSE (Life Technologies), or EASY-LYSE ERYTHROCYTE LYSIS SOLUTION (Dako). Additionally or in the alternative, erythrocytes can be lysed by using a lysis reagent that contains a fixative. For example, commercial reagents, such as FACSLYSE (BD Bioscience) or RBC LYSIS/FIXATION SOLUTION (BioLegend) can be used in place of ammonium chloride to lyse erythrocytes. However, as FACSLYSE contains a fixative, staining for cell surface markers on PBMCs have to be performed prior to lysis of the erythrocytes with this reagent. For example, whole blood samples can be stained with antibodies as needed, and then the erythrocytes can be lysed according to manufacturer's instructions. Additionally or in the alternative, OPTILYSE (Beckman Coulter) may be used to lyse erythrocytes without subsequent washing of the PBMCs.

In certain instances, PBMCs (e.g., lymphocytes, NK cells, etc.) can be isolated from the blood sample(s) by density gradient centrifugation (see, e.g., Boyum, “Isolation of mononuclear cells and granulocytes from human blood,” Scand J. Clin. Lab Invest. Suppl. 21:77-89 (1968); and Boyum, “Separation of lymphocytes, lymphocyte subgroups and monocytes: A review,” Lymphology 10:71-76 (1977)). Isolation of PBMCs by density gradient centrifugation can be useful when purification of cell populations is required rather than simple removal of erythroid contaminants. While the density gradient separation technique does not yield as many PBMCs as the simple lysis method, this method does have other distinct advantages. A key advantage of this method is the removal of most granulocytes from the sample. An additional advantage is the removal of nonviable cells from the sample. There are modifications of this method to enrich unlabeled cells of interest, such as by using ROSETTESEP TETRAMERIC ANTIBODY COMPLEXES (a cocktail of bi-specific antibodies to preferentially cross link undesired cells to RBCs; Stem Cell Technologies), which remove unwanted cells by crosslinking and pelleting together with RBCs. This leaves only the unlabeled cells of interest for studies, such as studies described herein. For isolation of PBMCs from blood sample(s) by density gradient centrifugation, a medium with a density of 1.077 g/mL can be used. For example, 1.077 g/mL Ficoll-Hypaque (GE Healthcare) or Histopaque-1077 (Sigma) or Lymphoprep (STEMCELL Technologies) can be used for isolation of PBMCs from blood sample(s) by density gradient centrifugation.

Density gradient separation methods for isolation of PBMCs are known in the art (see, e.g., Dagur and McCoy J P Jr., “Collection, Storage, and Preparation of Human Blood Cells,” Curr. Protoc. Cytom. 73:5.1.1-5.1.16 (2015)). In certain instances, anticoagulated blood can be diluted with an equal volume of PBS and layered over Ficoll-Hypaque solution. The layering can be done by gently pipetting the diluted blood down the side of the tube containing the Ficoll-Hypaque. Following centrifugation (e.g., for 30 min at 400×g, 22° C., with no brake), the PBMCs can be collected from the interface between the plasma (upper layer) and the Ficoll-Hypaque (bottom layer).

In certain instances, PBMCs isolated by the methods described hereinabove can be enriched for lymphocytes by depleting monocytoid cells from the mononuclear cell preparation, e.g., by letting the monocytoid cells adhere to a plastic tissue culture flask.

### Cell Viability Assay

Blood sample(s) and/or cells (e.g., PBMCs) isolated therefrom can be subjected to a dye exclusion test, wherein the blood sample(s) and/or cell suspension(s) are contacted with a cell viability dye to determine the number of viable cells. The dye exclusion test is based on the principle that live cells possess intact cell membranes that exclude certain dyes (e.g., a cell viability dye, such as trypan blue, eosin, propidium, etc.), whereas dead cells do not have intact cell membranes and thus fail to exclude these dyes. Thus, in this test, the blood sample(s) and/or cell suspension(s) is mixed with a cell viability dye and then visually examined, e.g., under a microscope, to determine whether the cells take up or exclude the dye. For example, a dye exclusion test can be done using trypan blue, wherein a viable cell will have a clear cytoplasm as intact cell membranes of the live cells would exclude the dye, whereas a nonviable cell would fail to exclude the dye and will appear to have a blue cytoplasm when examined under a microscope.

Incubation with Antigen

In some instances, blood sample(s) and/or cells (e.g., PBMCs) isolated therefrom is incubated with an antigen prior to further processing. For example, blood sample(s) and/or cells (e.g., PBMCs) isolated therefrom can be incubated with an antigen specific for an infection, such as COVID-19 (i.e., SARS-CoV2 infection), influenza, common cold, human immunodeficiency virus (HIV) infection, herpesvirus infection, hepatitits (e.g., hepatitis B virus infection, hepatitis C virus infection), norovirus infection, rotavirus infection, rabies virus infection, measles, mumps, rubella virus infection, chickenpox, pertussis, West Nile virus infection, meningitis, polio virus infection, Zika virus infection, cytomegalovirus infection, or enterovirus infection (e.g., coxsackievirus infection, echovirus infection). In certain instances, an antigen specific for an infection is a vaccine that is routinely used to prevent the infection. For example, blood sample(s) and/or PBMCs isolated therefrom can be incubated with a COVID-19 vaccine.

In certain instances, blood sample(s) and/or cells (e.g., PBMCs) isolated therefrom is/are incubated with an antigen for at least about 30 minutes (e.g., at least about 1 hour, at least about 2 hours, about least about 3 hours, at least about 4 hours, at least about 5 hours, at least about 6 hours, at least about 8 hours, at least about 12 hours, at least about 18 hours, at least about 24 hours, at least about 36 hours, at least about 48 hours, at least about 60 hours, at least about 3 days, at least about 4 days, at least about 5 days, at least about 6 days, or at least about 7 days, or about 1 hour to about 7 days, about 1 hour to about 6 days, about 1 hour to about 5 days, about 1 hour to about 4 days, about 1 hour to about 3 days, about 1 hour to about 60 hours, about 1 hour to about 48 hours, about 1 hour to about 36 hours, about 1 hour to about 24 hours, about 1 hour to about 18 hours, about 1 hour to about 12 hours, about 1 hour to about 8 hours, about 1 hour to about 6 hours, about 1 hour to about 5 hours, about 1 hour to about 4 hours, about 1 hour to about 3 hours, about 1 hour to about 2 hours, about 2 hours to about 7 days, about 2 hours to about 6 days, about 2 hours to about 5 days, about 2 hours to about 4 days, about 2 hours to about 3 days, about 2 hours to about 60 hours, about 2 hours to about 48 hours, about 2 hours to about 36 hours, about 2 hours to about 24 hours, about 2 hours to about 18 hours, about 2 hours to about 12 hours, about 2 hours to about 8 hours, about 2 hours to about 6 hours, about 2 hours to about 5 hours, about 2 hours to about 4 hours, about 2 hours to about 3 hours, about 3 hours to about 7 days, about 3 hours to about 6 days, about 3 hours to about 5 days, about 3 hours to about 4 days, about 3 hours to about 3 days, about 3 hours to about 60 hours, about 3 hours to about 48 hours, about 3 hours to about 36 hours, about 3 hours to about 24 hours, about 3 hours to about 18 hours, about 3 hours to about 12 hours, about 3 hours to about 8 hours, about 3 hours to about 6 hours, about 3 hours to about 5 hours, about 3 hours to about 4 hours, about 4 hours to about 7 days, about 4 hours to about 6 days, about 4 hours to about 5 days, about 4 hours to about 4 days, about 4 hours to about 3 days, about 4 hours to about 60 hours, about 4 hours to about 48 hours, about 4 hours to about 36 hours, about 4 hours to about 24 hours, about 4 hours to about 18 hours, about 4 hours to about 12 hours, about 4 hours to about 8 hours, about 4 hours to about 6 hours, about 4 hours to about 5 hours, about 5 hours to about 7 days, about 5 hours to about 6 days, about 5 hours to about 5 days, about 5 hours to about 4 days, about 5 hours to about 3 days, about 5 hours to about 60 hours, about 5 hours to about 48 hours, about 5 hours to about 36 hours, about 5 hours to about 24 hours, about 5 hours to about 18 hours, about 5 hours to about 12 hours, about 5 hours to about 8 hours, about 5 hours to about 6 hours, about 6 hours to about 7 days, about 6 hours to about 6 days, about 6 hours to about 5 days, about 6 hours to about 4 days, about 6 hours to about 3 days, about 6 hours to about 60 hours, about 6 hours to about 48 hours, about 6 hours to about 36 hours, about 6 hours to about 24 hours, about 6 hours to about 18 hours, about 6 hours to about 12 hours, about 6 hours to about 8 hours, about 8 hours to about 7 days, about 8 hours to about 6 days, about 8 hours to about 5 days, about 8 hours to about 4 days, about 8 hours to about 3 days, about 8 hours to about 60 hours, about 8 hours to about 48 hours, about 8 hours to about 36 hours, about 8 hours to about 24 hours, about 8 hours to about 18 hours, about 8 hours to about 12 hours, about 12 hours to about 7 days, about 12 hours to about 6 days, about 12 hours to about 5 days, about 12 hours to about 4 days, about 12 hours to about 3 days, about 12 hours to about 60 hours, about 12 hours to about 48 hours, about 12 hours to about 36 hours, about 12 hours to about 24 hours, about 12 hours to about 18 hours, about 18 hours to about 7 days, about 18 hours to about 6 days, about 18 hours to about 5 days, about 18 hours to about 4 days, about 18 hours to about 3 days, about 18 hours to about 60 hours, about 18 hours to about 48 hours, about 18 hours to about 36 hours, about 18 hours to about 24 hours, about 24 hours to about 7 days, about 24 hours to about 6 days, about 24 hours to about 5 days, about 24 hours to about 4 days, about 24 hours to about 3 days, about 24 hours to about 60 hours, about 24 hours to about 48 hours, about 24 hours to about 36 hours, about 36 hours to about 7 days, about 36 hours to about 6 days, about 36 hours to about 5 days, about 36 hours to about 4 days, about 36 hours to about 3 days, about 36 hours to about 60 hours, about 36 hours to about 48 hours, about 48 hours to about 7 days, about 48 hours to about 6 days, about 48 hours to about 5 days, about 48 hours to about 4 days, about 48 hours to about 3 days, about 48 hours to about 60 hours, about 60 hours to about 7 days, about 60 hours to about 6 days, about 60 hours to about 5 days, about 60 hours to about 4 days, about 60 hours to about 3 days, about 3 days to about 7 days, about 3 days to about 6 days, about 3 days to about 5 days, about 3 days to about 4 days, about 4 days to about 7 days, about 4 days to about 6 days, about 4 days to about 5 days, about 5 days to about 7 days, about 5 days to about 6 days, or about 6 days to about 7 days).

In certain instances, blood sample(s) and/or PBMCs isolated therefrom can be incubated with an antigen (e.g., antigen specific for an infection or cancer), so as to promote blast transformation of lymphocytes before preparation of a microscopic slide.

Labelling with Antibody

Blood sample(s) and/or cells (e.g., PBMCs) isolated therefrom can be contacted with (e.g., labelled with) one or more labelled antibodies (e.g., prior to further processing, e.g., preparation of a microscopic slide). The labelled antibodies can bind specifically to one or more cell surface proteins (e.g., cell surface antigens). For example, blood sample(s) and/or PBMCs isolated therefrom can be contacted with one or more labelled antibodies, where the labelled antibodies bind specifically to one or more cell surface antigens, such as CD3, CD4, CD5, CD8, CD11c, CD14, CD16, CD19, CD20, CD25, CD27, CD28, CD38, CD39, CD45, CD45RA, CD45RO, CD56, CD57, CD62L, CD66b, CD94, CD103, CD122, CD123, CD127, CD161, CD223, CD294, CCR4, CCR6, CCR7, CXCR3, CXCR5, HLA-DR, IgA, IgD, IgE, IgG, IgM, TCRγδ, KIR, NKG2A, NKGD, NKp30, NKp44, NKp46, NKp80, CTLA-4, GITR, IgA, IgD, IgE, IgG, and/or IgM. The labelled antibodies can help in immunophenotyping of blood sample(s) and/or cells (e.g., PBMCs) isolated therefrom, e.g., by microscopy (e.g., by immunofluorescent microscopy, immunohistochemical microscopy, immunoelectron microscopy etc.).

Antibodies for use in the present methods can be labelled with fluorophores (e.g., fluorescent labelling), enzymes (e.g., enzyme labelling), biotin, magnetic beads, agarose beads, magnetic agarose beads, and/or colloidal gold. For example, antibodies for use in the present methods can be labelled with fluorophores, such as hydroxycoumarin, aminocoumarin, methoxycoumarin, Cascade Blue, Pacific Blue, Pacific Orange, 3-hydroxyisonicotinaldehyde, Lucifer yellow, NBD ([2-(4-nitro-2,1,3-benzoxadiazol-7-yl)aminoethyl]trimethylammonium; NBD-TMA), R-Phycoerythrin (PE), PE-Cy5 conjugates (Cychrome, R670, Tri-Color, Quantum Red), PE-Cy7 conjugates, Red 613 (PE-Texas Red), PerCP (Peridinin chlorophyll protein), TruRed (PerCP-Cy5.5 conjugate), FluorX, fluorescein (FITC), BODIPY-FL, G-Dye100, G-Dye200, G-Dye300, G-Dye400, Cy2, Cy3, Cy3B, Cy3.5, Cy5, Cy5.5, Cy7, TRITC, X-Rhodamine, Lissamine, Rhodamine B, Texas Red, Allophycocyanin (APC), and/or APC-Cy7 conjugates (Far Red).

In some embodiments, fluorophore-labelled antibodies can be used for detection of cells (e.g., immunophenotyping of cells), such as by flow cytometry, immunofluorescence staining (e.g., for detection by fluorescent microscopy), or fluorescent Western assays. To detect a fluorescent label, an instrument is required that emits a specified wavelength of light that excites the fluorophore. The fluorescent dye then emits a signal in a different wavelength. The same instrument contains appropriate filters for detecting the emission from the fluorophore. Flow cytometry requires the use of a flow cytometer, while immunofluorescence (IF) uses a fluorescent microscope. A digital imaging system is usually employed for Western blots. Antibodies can be labeled with a variety of fluorescent dyes with varying excitation and emission spectra. In addition to being highly quantitative, fluorescent labels give the distinct advantage of being able to multiplex, or detect two or more different target proteins at the same time, through the use of dyes with non-overlapping emission spectra. For labelling, fluorescent tags (e.g., fluorophores) can be covalently attached to antibodies through primary amines or thiol groups.

Additionally, or in the alternative, antibodies for use in the present methods can be labelled with enzymes, such as horseradish peroxidase (HRP), alkaline phosphatase (AP), glucose oxidase, or β-galactosidase. Enzyme-labeled antibodies can be used for detection of cells (e.g., immunophenotyping of cells), such as by ELISA, Western blotting, and immunostaining. To use enzyme-labelled antibodies, samples (e.g., blood sample(s) and/or cells isolated therefrom) can be incubated with an enzyme-specific substrate that is catalyzed by the enzyme to produce a colored product (chromogenic assays) or light (chemiluminescent assays). Each enzyme has a set of substrates and detection methods that can be employed. For example, HRP can be reacted with diaminobenzidine to produce a brown-colored product, or with luminol to produce light. In contrast, AP can be reacted with para-nitrophenylphosphate (pNPP) to produce a yellow-colored product detected by a spectrophotometer or with 5-bromo-4-chloro-3-indolyl phosphate (BCIP) and nitroblue tetrazolium (NBT) to produce a purple colored precipitate. Color producing assays are useful for Western blots, ELISAs and immunohistochemistry, while light-producing reactions are most frequently used in Western blotting. Many substrates with varying sensitivities and outputs are available for detection, making enzyme reporters one of the most popular antibody labels.

Additionally, or in the alternative, antibodies for use in the present methods can be labelled with biotin. Biotin is a small molecule (244.3 Da) that forms one of the strongest non-covalent interactions found in nature with its binding partners, avidin (found in egg whites) and streptavidin (produced by the bacterium, Streptomyces avidini). Due to its size, biotin rarely disrupts the activity of antibodies, making it a good choice for a label. Biotin-labelled antibodies can be used for detection of cells (e.g., immunophenotyping of cells), such as by Western blot, ELISA, flow cytometry, immunofluorescence, and immunohistochemistry. Biotin-labelled antibodies are often used to increase the sensitivity of an assay when an antigen is difficult to detect. Blots or samples (e.g., blood sample(s) and/or cells isolated therefrom) can be incubated with biotin-labelled antibodies followed by a second incubation with avidin or streptavidin that is labelled with an enzyme or a fluorescent dye. Antibodies can be conjugated with multiple biotin molecules (3-6 molecules), leading to an amplification step that enhances detection of less abundant antigens.

### Preparation of Microscopic Slides

Blood sample(s) and/or cells (e.g., PBMCs) isolated therefrom can be deposited onto glass slides (e.g., microscopic slides) for evaluation. In certain instances, blood sample(s) and/or cells isolated therefrom can be contacted with (e.g., labelled with) one or more labelled antibodies prior to depositing the cells onto microscopic slides. Alternatively, microscopic slides prepared by the present methods can be stained with labelled antibodies described hereinabove. Microscopic slides for use in the present methods can be prepared by depositing blood sample(s) and/or cells onto the slides by techniques such as direct smear, touch preps, or filter techniques. Additionally, or in the alternative, microscopic slides for use in the present methods can be prepared by depositing blood sample(s) and/or cells onto the slides by using centrifugation, e.g., by Cytospin techniques. Cytospin preparations can be obtained by employing centrifugal force to isolate, concentrate, and deposit a monolayer of cells from a dilute cell suspension onto a circular area on a slide. The objective of Cytospin is to keep cells intact, thus enabling the morphology of the cells to be examined. Methods for Cytospin preparations have been described in the literature. See, e.g., Bibby, “Preparation of Cytospin Slides from Bloody Fluids,” Lab Med 17(4):228 (1986). Provided herein is an exemplary method for preparing microscopic slides by Cytospin. Briefly, 10⁵ cells (e.g., cells from blood sample(s)) can be washed in cold 2% FCS-PBS twice and dilute in 100 μL of cold 1% BSA-PBS. Slides and filters can be placed into appropriate slots in the Cytospin instrument with the cardboard filters facing the center of the cytospin. In the event that there are few cells available, about 100 μL of cold 1% BSA-PBS can be aliquoted into each of the wells and spun for 1-2 minutes, which would serve to wet the filter and allow more cells to reach the slide. About 100 μL of each sample can be aliquoted into the appropriate wells of the Cytospin and centrifuged at maximum speed for 1-3 minutes. The filters can then be removed from their slides without contacting the smears on the slides. The slides can be dried in a desiccation chamber overnight before analysis under microscope and/or before further processing. The slides can be fixed (e.g., with cold (4° C.) methanol) immediately after drying. In some instances, the slide can be covered with a coverslip, so as to prepare a permanent slide that can be stored and evaluated later.

Microscopic slides prepared by the methods described hereinabove can be examined (e.g., viewed and analyzed) under a microscope for determining the number of viable blast-transformed mononuclear cells and/or the number of viable large granular lymphocytes in the slides. The number or level of viable blast-transformed mononuclear cells and/or the number of viable large granular lymphocytes in the blood sample(s) can be determined based on the number of viable blast-transformed mononuclear cells and/or the number of viable large granular lymphocytes in the microscopic slides.

Assessing Prognosis of Cancer and/or Infection

A subject whose blood sample(s) (and/or cells prepared from the blood sample(s)) show an increased number or level of viable blast-transformed mononuclear cells and/or an increased number or level of viable large granular lymphocytes, as compared to a reference level (e.g., any of the exemplary reference levels described herein), may be identified as having improved prognosis of cancer and/or infection. For example, a subject having an increased number or level (e.g., at least a 1% increase, at least a 5% increase, at least a 10% increase, at least a 15% increase, at least a 20% increase, at least a 25% increase, at least a 30% increase, at least a 35% increase, at least a 40% increase, at least a 45% increase, at least a 50% increase, at least a 55% increase, at least a 60% increase, at least a 65% increase, at least a 70% increase, at least a 75% increase, at least a 80% increase, at least 85% increase, at least a 90% increase, at least a 95% increase, at least a 100% increase, at least a 120% increase, at least a 140% increase, at least a 160% increase, at least a 180% increase, at least a 200% increase, at least a 220% increase, at least a 240% increase, at least a 260% increase, at least a 280% increase, at least a 300% increase, at least a 320% increase, at least a 340% increase, at least a 360% increase, at least a 380% increase, or at least a 400% increase, or about a 1% increase to about a 400% increase (or any of the subranges of this range described herein)) of viable blast-transformed mononuclear cells and/or an increased number or level (e.g., at least a 1% increase, at least a 5% increase, at least a 10% increase, at least a 15% increase, at least a 20% increase, at least a 25% increase, at least a 30% increase, at least a 35% increase, at least a 40% increase, at least a 45% increase, at least a 50% increase, at least a 55% increase, at least a 60% increase, at least a 65% increase, at least a 70% increase, at least a 75% increase, at least a 80% increase, at least 85% increase, at least a 90% increase, at least a 95% increase, at least a 100% increase, at least a 120% increase, at least a 140% increase, at least a 160% increase, at least a 180% increase, at least a 200% increase, at least a 220% increase, at least a 240% increase, at least a 260% increase, at least a 280% increase, at least a 300% increase, at least a 320% increase, at least a 340% increase, at least a 360% increase, at least a 380% increase, or at least a 400% increase, or about a 1% increase to about a 400% increase (or any of the subranges of this range described herein)) of viable large granular lymphocytes, as compared to a reference level (e.g., any of the exemplary reference levels described herein), may be identified as having an improved prognosis of cancer and/or infection.

A subject with a decreased number or level of viable blast-transformed mononuclear cells and/or a decreased number or level of viable large granular lymphocytes, as compared to a reference level (e.g., any of the exemplary reference levels described herein), may be identified as having poor prognosis of one or more cancers and/or infections. For example, a subject having a decreased number or level (e.g., at least a 1% decrease, at least a 5% decrease, at least a 10% decrease, at least a 15% decrease, at least a 20% decrease, at least a 25% decrease, at least a 30% decrease, at least a 35% decrease, at least a 40% decrease, at least a 45% decrease, at least a 50% decrease, at least a 55% decrease, at least a 60% decrease, at least a 65% decrease, at least 70% decrease, at least a 75% decrease, at least a 80% decrease, at least a 85% decrease, at least a 90% decrease, at least a 95% decrease, or at least a 99% decrease, or about a 1% decrease to about a 99% decrease (or any of the subranges of this range described herein)) of viable blast-transformed mononuclear cells and/or a decreased number or level (e.g., at least a 1% decrease, at least a 5% decrease, at least a 10% decrease, at least a 15% decrease, at least a 20% decrease, at least a 25% decrease, at least a 30% decrease, at least a 35% decrease, at least a 40% decrease, at least a 45% decrease, at least a 50% decrease, at least a 55% decrease, at least a 60% decrease, at least a 65% decrease, at least 70% decrease, at least a 75% decrease, at least a 80% decrease, at least a 85% decrease, at least a 90% decrease, at least a 95% decrease, or at least a 99% decrease, or about a 1% decrease to about a 99% decrease (or any of the subranges of this range described herein)) of viable large granular lymphocytes, as compared to a reference level (e.g., any of the exemplary reference levels described herein), may be identified as having poor prognosis of cancer and/or infection.

In certain instances, a reference level is about the 70th percentile, about the 71st percentile, about the 72nd percentile, about the 73rd percentile, about the 74th percentile, about the 75th percentile, about the 76th percentile, about the 77th percentile, about the 78th percentile, about the 79th percentile, about the 80th percentile, about the 81st percentile, about the 82nd percentile, about the 83rd percentile, about the 84th percentile, about the 85th percentile, about the 86th percentile, about the 87th percentile, about the 88th percentile, about the 89th percentile, about the 90th percentile, about the 91st percentile, about the 92nd percentile, about the 93rd percentile, about the 94th percentile, about the 95th percentile, about the 96th percentile, about the 97th percentile, about the 98th percentile, or about the 99th percentile)) of the median level of the number or level of viable blast-transformed mononuclear cells or the number or level of viable large granular lymphocytes in a healthy patient population. A healthy patient population may be a patient population that is not currently diagnosed with a cancer and/or an infection, or has not been diagnosed with a cancer and/or an infection within the last 5 years (e.g., within the last 4 years, 3 years, 2 years, 1 year, 11 months, 10 months, 9 months, 8 months, 7 months, 6 months, 5 months, 4 months, 3 months, 2 months, 1 month, 3 weeks, 2 weeks, or 1 week).

A subject identified as having an improved or poor prognosis of cancer and/or infection by the present methods can be treated accordingly. For example, a subject identified as having an improved prognosis of cancer and/or infection can be selected for a low frequency of cancer and/or infection screening. A low frequency of screening may comprise one screening every 1 year, every 2 years, every 3 years, every 4 years, every 5 years, every 6 years, every 7 years, every 8 years, every 9 years, or every 10 years, or more. A subject identified as having a poor prognosis of cancer and/or infection can be selected for a high frequency of cancer and/or infection screening. A high frequency of screening may comprise one screening every 1 day, every 2 days, every 3 days, every 4 days, every 5 days, every 6 days, every 7 days, every 1 week, every 2 weeks, every 3 weeks, every 4 weeks, every 1 month, every 2 months, every 3 months, every 4 months, every 5 months, every 6 months, every 7 months, every 8 months, every 9 months, every 10 months, or every 11 months. A high frequency of screening may comprise at least two screenings a year, at least three screenings a year, at least four screenings a year, at least five screenings a year, at least six screenings a year, at least seven screenings a year, at least eight screenings a year, at least nine screenings a year, at least ten screenings a year, at least eleven screenings a year, at least twelve screenings a year, at least 24 screenings a year, at least 52 screenings a year (e.g., weekly screenings), or at least 365 screenings a year (e.g., daily screenings).

### Subject

As used herein, the term “subject” can refer to human and non-human animals. Non-human animals can include vertebrates, e.g., mammals and non-mammals, such as non-human primates, sheep, cats, horses, cows, chickens, dog, mouse, rat, goat, rabbit, and pig. For example, the subject can be a human.

In certain instances, the subject is not identified or diagnosed as having a cancer and/or an infection. For example, the subject may not be currently (or recently) identified or diagnosed as having a cancer and/or an infection, such as not identified or diagnosed as having a cancer and/or an infection within the last 5 years (e.g., within the last 4 years, within the last 3 years, within the last 2 years, within the last 1 year, within the last 11 months, within the last 10 months, within the last 9 months, within the last 8 months, within the last 7 months, within the last 6 months, within the last 5 months, within the last 4 20 months, within the last 3 months, within the last 2 months, within the last 1 month, within the last 3 weeks, within the last 2 weeks, or within the last 1 week).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of determining prognosis of cancer or infection in a subject, the method comprising:
(a) contacting a blood sample obtained from the subject with a cell viability dye;
(b) after step (a), generating a microscopic slide using the blood sample;
(c) identifying a number of viable blast-transformed mononuclear cells and/or a number of viable large granular lymphocytes in the microscopic slide using microscopic analysis; and
(d) identifying a subject having an increased number of viable blast-transformed mononuclear cells and/or an increased number of viable large granular lymphocytes, as compared to a reference level, as having an improved prognosis of cancer or infection; or
identifying a subject having a decreased number of viable blast-transformed mononuclear cells and/or a decreased number of viable large granular lymphocytes, as compared to a reference level, as having a poor prognosis of cancer or infection.

2. The method of claim 1, wherein step (b) comprises one or more centrifugation steps.

3. The method of claim 2, wherein one of the centrifugation steps comprises the use of a Cytospin instrument.

4. (canceled)

5. The method of claim 1, wherein step (c) comprises identifying the number of viable blast-transformed mononuclear cells in the microscopic slide.

6. The method of claim 1, wherein step (c) comprises identifying the number of viable large granular lymphocytes in the microscopic slide.

7. The method of claim 1, wherein step (c) comprises identifying the number of viable blast-transformed mononuclear cells and the number of viable large granular lymphocytes in the microscopic slide.

8.-10. (canceled)

11. The method of claim 1, wherein step (b) comprises the use of one or more fluorophore-labelled antibodies specific for one or more cell surface proteins.

12. The method of claim 11, wherein the one or more labelled antibodies bind specifically to an antigen selected from the group consisting of: CD3, CD4, CD5, CD8, CD11c, CD14, CD16, CD19, CD20, CD25, CD27, CD28, CD38, CD39, CD45, CD45RA, CD45RO, CD56, CD57, CD62L, CD66b, CD94, CD103, CD122, CD123, CD127, CD161, CD223, CD294, CCR4, CCR6, CCR7, CXCR3, CXCR5, IgA, IgD, IgE, IgG, IgM, TCRγδ, KIR, NKG2A, NKGD, NKp30, NKp44, NKp46, NKp80, CTLA-4, and GITR.

13. (canceled)

14. The method of claim 1, wherein before step (b), the method further comprises incubating the blood sample with an antigen specific for an infection for 6 hours to 3 days.

15. (canceled)

16. The method of claim 1, wherein the viable blast-transformed mononuclear cells are viable blast transformed T cells and/or viable blast transformed B cells.

17. The method of claim 1, wherein the viable large granular lymphocytes are NK cells.

18. The method of claim 1, wherein the blood sample is a venous blood sample.

19. The method of claim 1, wherein the blood sample is a capillary blood sample.

20. The method of claim 19, wherein the capillary blood sample is a finger stick blood sample.

21. The method of claim 1, wherein the method further comprises generating a frozen blood sample from a portion of the blood sample.

22.-25. (canceled)

26. The method of claim 21, wherein the method further comprises thawing the frozen blood sample and performing additional immunological testing/analysis on the thawed blood sample.

27.-28. (canceled)

29. The method of claim 1, wherein the method comprises selecting for a subject identified as having an improved prognosis of cancer or infection a low frequency of cancer or infection screening for the subject.

30. The method of claim 1, wherein the method comprises selecting for a subject identified as having a poor prognosis of cancer or infection a high frequency of cancer or infection screening for the subject.

31. A method of selecting a treatment for a subject not identified or diagnosed as having cancer or infection, the method comprising:
(a) contacting a blood sample obtained from the subject with a cell viability dye;
(b) after step (a), generating a microscopic slide using the blood sample;
(c) identifying a number of viable blast-transformed mononuclear cells and/or a number of viable large granular lymphocytes in the microscopic slide using microscopic analysis;
(d) identifying a subject having an increased number of viable blast-transformed mononuclear cells and/or an increased number of viable large granular lymphocytes, as compared to a reference level, as having an improved prognosis of cancer or infection; or identifying a subject having a decreased number of viable blast-transformed mononuclear cells and/or a decreased number of viable large granular lymphocytes, as compared to a reference level, as having a poor prognosis of cancer or infection; and
(e) selecting for a subject identified as having an improved prognosis of cancer or infection a low frequency of cancer or infection screening for the subject, or selecting for a subject identified as having a poor prognosis of cancer or infection a high frequency of cancer or infection screening for the subject.

32.-58. (canceled)

59. A method of treating a subject not identified as having cancer or infection, the method comprising:
(a) contacting a blood sample obtained from the subject with a cell viability dye;
(b) after step (a), generating a microscopic slide using the blood sample;
(c) identifying a number of viable blast-transformed mononuclear cells and/or a number of viable large granular lymphocytes in the microscopic slide using microscopic analysis;
(d) identifying a subject having an increased number of viable blast-transformed mononuclear cells and/or an increased number of viable large granular lymphocytes, as compared to a reference level, as having an improved prognosis of cancer or infection; or identifying a subject having a decreased number of viable blast-transformed mononuclear cells and/or a decreased number of viable large granular lymphocytes, as compared to a reference level, as having a poor prognosis of cancer or infection; and
(e) performing low frequency cancer or infection screening on a subject identified as having an improved prognosis of cancer or infection, or performing high frequency cancer or infection screening on a subject identified as having a poor prognosis of cancer or infection.

60.-86. (canceled)
